SEQUENCE LISTING (36 sequences)

SEQ ID NO: 1 - DNA, 20 bp, synthetic construct
  source 1..20: mol_type = other DNA
gagtcaacggatttggtcgt

SEQ ID NO: 2 - DNA, 20 bp, synthetic construct
  source 1..20: mol_type = other DNA
ttgattttggagggatctcg

SEQ ID NO: 3 - DNA, 20 bp, synthetic construct
  source 1..20: mol_type = other DNA
acgacgatgacaagacggaa

SEQ ID NO: 4 - DNA, 19 bp, synthetic construct
  source 1..19: mol_type = other DNA
atggcgctgtactcctcct

SEQ ID NO: 5 - DNA, 20 bp, synthetic construct
  source 1..20: mol_type = other DNA
actaacttcccagcaggaat

SEQ ID NO: 6 - DNA, 20 bp, synthetic construct
  source 1..20: mol_type = other DNA
gttggtcacttgtttctcca

SEQ ID NO: 7 - DNA, 24 bp, synthetic construct
  source 1..24: mol_type = other DNA
cctgcgaccttaagatttgtgatt

SEQ ID NO: 8 - DNA, 24 bp, synthetic construct
  source 1..24: mol_type = other DNA
cagggaagatgggccggttagaga

SEQ ID NO: 9 - DNA, 20 bp, synthetic construct
  source 1..20: mol_type = other DNA
tgacatccggttcgtctaca

SEQ ID NO: 10 - DNA, 20 bp, synthetic construct
  source 1..20: mol_type = other DNA
tgcagttttccagcaatgag

SEQ ID NO: 11 - DNA, 30 bp, synthetic construct
  source 1..30: mol_type = other DNA
atgattccctttttacccatgttttctcta

SEQ ID NO: 12 - DNA, 30 bp, synthetic construct
  source 1..30: mol_type = other DNA
gaaggaataatcatgccattttttaagtcc

SEQ ID NO: 13 - DNA, 23 bp, synthetic construct
  source 1..23: mol_type = other DNA
cttggtgaccatctcagaactgg

SEQ ID NO: 14 - DNA, 22 bp, synthetic construct
  source 1..22: mol_type = other DNA
cttctgctcctgtgagttcacg

SEQ ID NO: 15 - DNA, 22 bp, synthetic construct
  source 1..22: mol_type = other DNA
ccaatgcctacgacaagacagc

SEQ ID NO: 16 - DNA, 22 bp, synthetic construct
  source 1..22: mol_type = other DNA
tgggaagtgacctcgtttgcca

SEQ ID NO: 17 - DNA, 22 bp, synthetic construct
  source 1..22: mol_type = other DNA
gcagcactacttcttgaccacc

SEQ ID NO: 18 - DNA, 22 bp, synthetic construct
  source 1..22: mol_type = other DNA
tctgctcctgagcattgacgtc

SEQ ID NO: 19 - DNA, 20 bp, synthetic construct
  source 1..20: mol_type = other DNA
cgcttttgggtacatggagt

SEQ ID NO: 20 - DNA, 20 bp, synthetic construct
  source 1..20: mol_type = other DNA
ttgcagcaaggtttcacaag

SEQ ID NO: 21 - DNA, 22 bp, synthetic construct
  source 1..22: mol_type = other DNA
tggactacctgcactcggagaa

SEQ ID NO: 22 - DNA, 22 bp, synthetic construct
  source 1..22: mol_type = other DNA
gtgccgcaaaaggtcttcatgg

SEQ ID NO: 23 - DNA, 22 bp, synthetic construct
  source 1..22: mol_type = other DNA
cagagcaactctgccaccagta

SEQ ID NO: 24 - DNA, 23 bp, synthetic construct
  source 1..23: mol_type = other DNA
catttgtggcaggaacttgctcc

SEQ ID NO: 25 - DNA, 21 bp, synthetic construct
  source 1..21: mol_type = other DNA
gtggcaggacatgaagaagaa

SEQ ID NO: 26 - DNA, 20 bp, synthetic construct
  source 1..20: mol_type = other DNA
catcagcacagaggcaaaga

SEQ ID NO: 27 - DNA, 22 bp, synthetic construct
  source 1..22: mol_type = other DNA
ctcatctactcccaggttctct

SEQ ID NO: 28 - DNA, 20 bp, synthetic construct
  source 1..20: mol_type = other DNA
gttgaccttgttcgggtagg

SEQ ID NO: 29 - DNA, 22 bp, synthetic construct
  source 1..22: mol_type = other DNA
gtcaactgcatgaacagaaagg

SEQ ID NO: 30 - DNA, 21 bp, synthetic construct
  source 1..21: mol_type = other DNA
agcaggcaggtctcattattc

SEQ ID NO: 31 - DNA, 22 bp, synthetic construct
  source 1..22: mol_type = other DNA
cgaacccaagctacaggaatag

SEQ ID NO: 32 - DNA, 21 bp, synthetic construct
  source 1..21: mol_type = other DNA
tggaaagtgtgtgtccaatca

SEQ ID NO: 33 - DNA, 22 bp, synthetic construct
  source 1..22: mol_type = other DNA
cctgtgggatttgagtgtctta

SEQ ID NO: 34 - DNA, 22 bp, synthetic construct
  source 1..22: mol_type = other DNA
gctcggcttaggagttagaaag

SEQ ID NO: 35 - DNA, 23 bp, synthetic construct
  source 1..23: mol_type = other DNA
ggtgacatccacgactactttag

SEQ ID NO: 36 - DNA, 21 bp, synthetic construct
  source 1..21: mol_type = other DNA
ccaggcatagctgttgtactt

(19) United States
(12) Patent Application Publication
(10) Pub. No.: US 2026/0124243 A1
(43) Pub. Date: May 7, 2026

(54) COMPOSITIONS AND METHODS FOR WOUND HEALING

(71) Applicants: RION INC., Rochester, MN (US); MAYO FOUNDATION FOR MEDICAL EDUCATION AND RESEARCH, Rochester, MN (US)

(72) Inventors: Atta Behfar, Rochester, MN (US); Steven L. Moran, Rochester, MN (US); Brooke Paradise, Rochester, MN (US); Laura Becher, Rochester, MN (US); Christopher Paradise, Mantorville, MN (US)

(21) Appl. No.: 18/704,240

(22) PCT Filed: Oct. 25, 2022
(86) PCT No.: PCT/US2022/047721

§ 371 Date: Apr. 24, 2024

Related U.S. Application Data
(60) Provisional application No. 63/271,486, filed on Oct. 25, 2021.

(51) Int. Cl. A61K 35/19; A61K 9/06; A61K 38/18; A61K 47/42; A61P 17/02

(52) U.S. Cl. CPC A61K 35/19; A61K 9/06; A61K 38/1841; A61K 47/42; A61P 17/02

(57) ABSTRACT

A method of promoting healing of a wound generally includes administering to the wound an amount of a PEP exosome preparation effective to promote healing of the wound. In one or more embodiments, the PEP exosome preparation includes a tissue sealant or surgical glue. In one or more embodiments, the wound is an ischemic wound, a puncture wound, a laceration, an abrasion, a surgical wound, a skin graft, or a traumatic wound.

### CROSS-REFERENCE TO RELATED APPLICATION

This application claims the benefit of U.S. Provisional Patent Application No. 63/271,486, filed Oct. 25, 2021, which is incorporated herein by reference in its entirety.

### SEQUENCE LISTING

This application contains a Sequence Listing electronically submitted to the United States Patent and Trademark Office via Patent Center as an XML file entitled “0560-000014WO01” having a size of 31.8 kilobytes and created on Oct. 25, 2022. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a method of promoting healing of a wound. Generally, the method includes administering to the wound an amount of a PEP preparation effective to promote healing of the wound.

In one or more embodiments, the PEP preparation includes a hydrogel that includes a basement membrane protein.

In one or more embodiments, the PEP preparation includes a hydrogel that includes a thrombin sealant or a fibrin sealant.

In one or more embodiments, the wound is an ischemic wound, a puncture wound, a laceration, an abrasion, a surgical wound, a skin graft, or a traumatic wound.

In one or more embodiments, the amount of PEP preparation administered to the wound is effective to increase angiogenesis, increase migration of fibroblasts into the wound, or increase migration of keratinocytes into the wound compared to a comparable untreated wound.

In one or more embodiments, the amount of PEP preparation administered to the wound is effective to donate TGF-β to increase expression COL1A or COL3A compared to a comparable untreated wound.

In one or more embodiments, the amount of PEP preparation administered to the wound is effective to decrease Wagner Ulcer Classification grade of the wound compared to a comparable untreated wound.

In one or more embodiments, the amount of PEP preparation administered to the wound is effective to decrease reaction force variation (R_{c}) or increase resistance to tensile force compared to a comparable untreated wound.

In one or more embodiments, the amount of PEP preparation administered to the wound is effective to increase expression of SMAD2, RAS, MKK3, RHOA, P38, or periostin in keratinocytes compared to untreated keratinocytes.

In one or more embodiments, the amount of PEP preparation administered to the wound is effective to increase expression of SMAD2, RAS, MKK3, ERK1, or TAK1 in fibroblasts compared to untreated fibroblasts.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. PEP extracellular vesicles display exosomal characteristics. (A) A representative transmission electron microscopic image of PEP exosomes. Scale bar, 200 nm. (B) Representative western blot of CD63, CD9, and Alix in PEP exosomes. GAPDH was used as a loading control. (C) Size distribution of PEP exosomes as measured by Nanoparticle Tracking Analysis (NTA), peaking at a diameter of 105.4 nm. (D) Size distribution of PEP exosomes as measured by nano flow cytometry (NanoFCM), with a mean of 123.49 nm.

FIG. 2. PEP extracellular vesicles display exosomal surface markers. (A) Representative simple western blot (Jess, ProteinSimple) shows the presence of CD41 (platelet marker), CD9, CD63, and Flotillin-1 (EV markers) in PEP. (B) Representative plot from nano flow cytometry of PEP demonstrates the presence of CD41a (platelet marker) on PEP extracellular vesicles (MemGlow488+, lipid bilayer stain). (C) Pie chart showing affinity-based capture of CD41a+ vesicles with subsequent fluorescent antibody staining of CD9, CD63, or CD81 surface markers (NanoView). (D) Bar graph showing affinity-based capture of CD41a+ vesicles with subsequent fluorescent antibody staining of CD9, CD63, or CD81 surface markers (NanoView).

FIG. 3. PEP microvesicles promote angiogenesis in vitro. (A) In vitro angiogenesis assay using co-culture of human dermal fibroblast (HDFB) with GFP-tagged human umbilical vascular endothelial cells (HUVEC) in presence of VEGF, PEP, or suramin (angiogenesis inhibitor). Scale bar, 200 μm. (B) Graph displaying quantification of tube formation in six hour increments. (C) 3D organoid differentiation assay of human keratinocytes treated with PEP or serum free media. Collection of organoids was performed at day 24 and sections were prepped for microscopy. Hematoxylin and eosin (H&E) staining was performed on each group. Upper panel arrows: Organized differentiated keratinocyte with multiple epidermis-like layers. Lower panel arrow: unorganized keratinocytes differentiation. (D) Brightfield microscopy images showing in vitro angiogenesis assay culture of HUVECs on extracellular matrix coated plates in serum-free or PEP conditions. (E) Quantitation of tube network formation in in vitro angiogenesis assay culture of HUVECs on extracellular matrix coated plates in serum-free, FBS, or PEP conditions.

FIG. 4. PEP microvesicles promote wound healing in vitro. (A) Scratch assay evaluating the migration of primary fibroblast treated with FBS, PEP, or serum free media. Representative pictures of wound closure for FBS vs. PEP vs. serum free media at 0 hours and 32 hours. (B) Graph showing quantification of fibroblast wound closure as determined via microscopy imaging performed every two hours. (C) Scratch assay testing the migration of human keratinocytes treated with FBS, PEP, or serum free media. Representative pictures of wound closure for FBS vs. PEP vs. serum free at 0 hours and 72 hours. (D) Graph showing quantification of keratinocytes wound closure as determined via microscopy imaging performed every three hours. ***p<0.001, ****p<0.0001.

FIG. 5. PEP microvesicles stimulate TGF-β-mediated wound healing in vitro. (A) Schematic illustration of the mechanism of PEP-induced wound healing in vitro. (B) Representative western blot of TGF-β in PEP exosomes. GAPDH was used as a loading control. (C) ELISA-based analysis of TGF-β concentration in four different lots of PEP (Ella, ProteinSimple). (D) Pro-collagen I protein concentration (ELISA) in PEP-treated fibroblast. (E) Pro-collagen III protein concentration (ELISA) in PEP-treated fibroblast. **p<0.01.

FIG. 6. PEP microvesicles stimulate TGF-β-mediated wound healing in vitro. (A) Quantification of Smad2, Ras, MKK3, RhoA, P38, and periostin mRNA expression in PEP-treated keratinocytes. (B) Quantification of Smad2, Ras, MKK3, Erk1, and TAK1 mRNA expression in PEP-treated fibroblasts. A 2-tailed unpaired Student's t-test was used for each group compared to the untreated control group. *p<0.05, **p<0.01.

FIG. 7. PEP is eluted from PEP-TISSEEL biogel over time. (A) Representative scanning electron microscopy (SEM) photo of TISSEEL alone vs TISSEEL+PEP, showing that PEP binds fibrils in TISSEEL. (B) PEP extracellular vesicle concentration eluted from TISSEEL over seven days in an in vitro elution assay quantified by Nanoparticle Tracking Analysis (NTA, Nanosight NS300). (C) Mean PEP extracellular vesicle size eluted from TISSEEL over seven days, quantified by NTA (Nanosight).

FIG. 8. TISSEEL-PEP biogel promotes cell migration in a scratch assay. (A) Representative brightfield microscopy images of MSC migration towards PEP-TISSEEL biogel at Time=0. (B) Representative brightfield microscopy images of MSC migration towards PEP-TISSEEL biogel at four hours. (C) Representative brightfield microscopy images of MSC migration towards PEP-TISSEEL biogel at 21 hours. (D) Representative brightfield microscopy images of MSC migration towards PEP-TISSEEL biogel at 48 hours. (E) Representative brightfield microscopy images of MSC migration towards PEP-TISSEEL biogel at 144 hours. (F) Scratch area quantified from brightfield microscopy images.

FIG. 9. TISSEEL-PEP biogel promotes ischemic wound healing in vivo. (A) Schematic of rabbit ischemic ear punch biopsy wound model. Ligation of arteries produced an ischemic wound environment. Animals were divided into four groups. Skin defects were left untreated, treated with PEP alone, TISSEEL (Baxter International, Inc., Deerfield, IL) alone, or the PEP-TISSEEL biogel. (B) Photographs of representative wounds from each of four groups.

FIG. 10. TISSEEL-PEP biogel promotes ischemic wound healing in vivo. (A) Bar graphs showing quantification of wound healing (FIG. 9). Each bar measures average wound size for each group as a percentage of original wound on day 28. (B) Oil level of skin tissue four weeks post injury. (C) Hydration of skin tissue four weeks post injury. The skin hydration and oil level of different treated groups were measured and compared with the untreated group. Normal skin served as baseline. (D) Clinical assessment of wound closure. All groups were assessed by a board certified physician using the Wagner Ulcer Classification system each week post injury. Each individual data point was plotted in the graph with a smoothing spline curve created. ***p<0.001.

FIG. 11. PEP contributes to structural reorganization in wound tissue. (A) Hematoxylin and eosin (H&E) staining analysis was performed on untreated, TISSEEL only control, PEP only, TISSEEL-PEP, and normal skin. Tissue samples were collected for analysis at the time of sacrifice 28 days post injury. Representative images from each group shown at two magnifications. Scale bar in normal skin column represents 100 μm. ▴: unhealed area. Yellow arrow: hair follicle. Red arrow: new blood vessel.

FIG. 12. PEP contributes to structural reorganization in wound tissue. H&E stained tissue sections collected from the wound site (FIG. 11) were analyzed using Image J software. (A) Quantification of epidermal layer thickness 28 days after treatment performed in 10 different locations per slide representatively. N=4. (B) Quantification of dermal layer thickness 28 days after treatment performed in five different locations per slide representatively. N=4. ***p<0.001.

FIG. 13. PEP contributes to structural reorganization in wound tissue. Representative 3D-electron microscopy reconstruction of wound tissue (n=3) collected at time of sacrifice 28 days after treatment. Yellow arrow: fibroblast. Red arrow: disorganized collagen deposition. Colored Region: new capillary with red blood cell. Reference bar in normal skin is 1 m.

FIG. 14. PEP biogel activated TGF-3 signaling and promoted collagen organization. Masson Trichrome staining analysis (top row), TGF-β immunofluorescence staining (second row), Col1A immunofluorescence staining (third row), Col3A staining (fourth row), and Col1A/Col3A combined staining of untreated, TISSEEL only control, PEP only, TISSEEL-PEP, and normal skin. Skin tissue was obtained at day 28 post surgery. Scale bar, 200 μm.

FIG. 15. PEP biogel activated TGF-β signaling and promoted collagen organization. (A) Quantification of immunofluorescence staining for TGF-β. (B) Quantification of immunofluorescence staining for Col1A, Col3A, and the calculated ratio of Col3A:Col1A. (C) Cyclic tensile test for all the groups. Untreated and TISSEEL-treated only groups were stiffer and less like normal skin. Rc=reaction force variation. (D) Maximum tensile test of all the groups. The PEP-TISSEEL group had skin that could resist the highest tensile forces.

FIG. 16. TISSEEL-PEP treatment mediated transcriptional changes of genes that promote wound healing events. mRNA was isolated from tissue biopsies of the wound site collected 28 days post-treatment and analyzed using RNA sequencing (RNA-seq). Tertiary analysis of resulting gene expression data demonstrated differentially expressed genes in wound tissue collected from TISSEEL only (left three columns) compared to TISSEEL-PEP (right three columns) treated groups (N=3, genes were filtered with normalized counts of 100, filtered genes met the criteria of |log₂FC|>0.5 and p<0.05 were considered significantly changed). Heatmap demonstrates differentially upregulated (red) and downregulated (blue) genes represented as a fold change to the untreated control.

FIG. 17. TISSEEL-PEP treatment mediated transcriptional changes of genes related to pro-wound healing events. Gene ontology and pathway analysis of significantly differentially expressed genes as determined by RNA sequencing analysis. The top Upregulated and Downregulated 10 pathways were shown (p<0.05). Most significant and nonredundant Biological Process or Pathways with respective gene number and p-value are shown. (A) Heatmaps of differentially regulated genes involved in extracellular structure organization. (B) Heatmaps of differentially regulated genes involved in regulation of angiogenesis. (C) Heatmaps of differentially regulated genes involved in skin development. (D) Heatmaps of differentially regulated genes involved in VEGF signaling. (E) Heatmaps of differentially regulated genes involved in response to wounding. (F) Heatmaps of differentially regulated genes involved in collagen metabolic processes. (G) Heatmaps of differentially regulated genes involved in positive regulation of cell cycle. (H) Heatmaps of differentially regulated genes involved in regulation of NIK/NF-KB signaling. Heatmaps demonstrate differentially upregulated (red) and downregulated (blue) genes represented as a fold change to the untreated control.

### DETAILED DESCRIPTION OF ILLUSTRATIVE EMBODIMENT

This disclosure describes using a platelet-derived clinical-grade exosome product (PEP), enriched with TGF-β (Transforming Growth Factor Beta), formulated as a lyophilized off-the-shelf regenerative platform for wound treatment. While described below in an exemplary context of treating ischemic wounds, the compositions and methods described herein can include treatment of any type of wound including, but not limited to, an ischemic wound (e.g., an ischemic ulcer), a puncture wound, a laceration, an abrasion, a surgical wound, a skin graft, a thermal burn wound, a radiation exposure induced wound or a traumatic wound.

Topical treatment of ischemic wounds with PEP, carried in a fibrin sealant (TISSEEL, Baxter International, Inc., Deerfield, IL), promoted full-thickness healing with the reacquisition of hair follicles and sebaceous glands. Superior to untreated and TISSEEL-only treated controls, TISSEEL-PEP drove cutaneous healing associated with collagen synthesis and restoration of dermal architecture. Furthermore, PEP promoted epithelial and vascular cell activity enhancing angiogenesis to restore blood flow and mature skin function. Transcriptome deconvolution of TISSEEL-PEP versus TISSEEL-only treated wounds prioritized regenerative pathways encompassing neovascularization, matrix remodeling, and tissue growth. The composition is stable at room temperature when lyophilized and can therefore provide a bioactive growth factor to drive regenerative events.

Ischemic wounds affect millions of patients globally, precipitating life-threatening amputations and severe morbidity. For example, chronic ischemic wounds can progress to limb amputation with an associated 57% five-year mortality. Wound development is attributed to compromised cellular proliferation, impaired angiogenesis, and limited epithelization. Current management includes wound dressing, topical medications, and surgery. However, no treatment to date has achieved restoration of normal skin architecture. To improve outcomes, cell-based therapies are increasingly considered as an adjunct to standard-of-care. Lacking ease-of-use and impeded by high cost, their utility remains limited, warranting development of feasible and widely accessible regenerative alternatives.

Extracellular vesicles (EVs) and their exosome subsets offer a next-generation scalable option for wound healing. Exosomes have been shown to promote healing through angiogenesis, cell proliferation and migration, and ultimately re-epithelialization. Transferrable through the cell membrane to mediate cell-cell communication, exosomes are highly uniform cell-secreted vesicles ranging from 30-150 nm in diameter, capable of shuttling lipid-encapsulated signaling proteins and nucleotides between cells.

PEP is fully characterized and methods for preparing PEP are described in International Patent Application NO. PCT/US2018/065627 (published as International Publication No. WO 2019/118817), which is incorporated by reference herein in its entirety. Briefly, PEP typically has a spherical or spheroidal structure rather than a crystalline structure. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with the majority of the exosome structures having a mean diameter of 110 nm±50 nm such as, for example, 110 nm±30 nm.

This disclosure describes the use of a PEP preparation that is CD63+, CD9+, Alix-positive and prepared from activated platelets. The PEP preparation accelerated wound healing by releasing bioactive TGF-β into the wound bed. Sustained delivery through use of a fibrin sealant yielded significant regenerative benefit with full-thickness ischemic wound healing. The results herein provide first evidence of the ability to preserve TGF-β bioactivity in a lyophilized exosome product applied to accelerate wound healing.

Extracellular Vesicles Contained within PEP Display Exosomal Traits

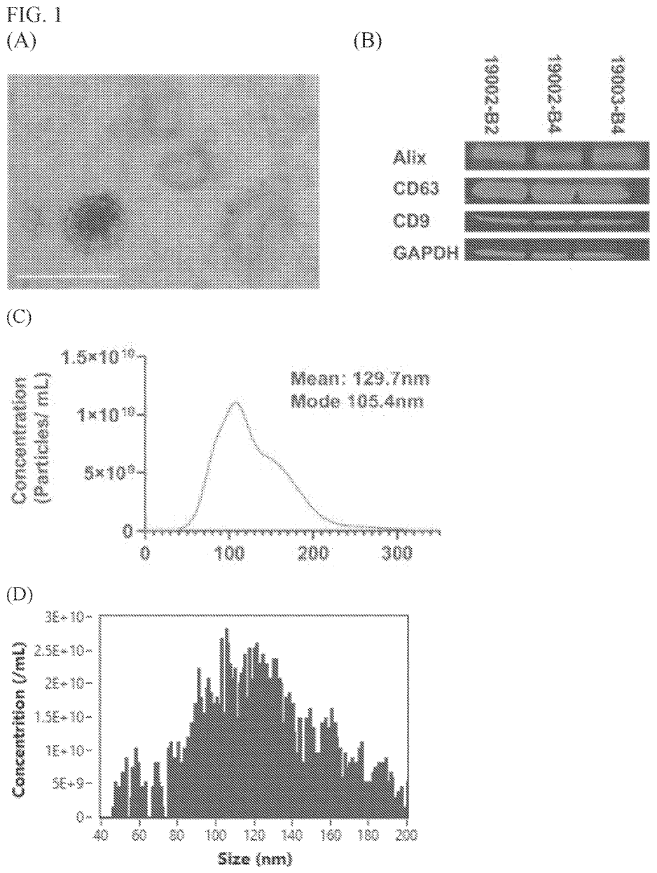

To ensure uniformity in cGMP regenerative exosomes, PEP vesicles were evaluated for vesicular morphology, exosomal surface markers, and analyzed using nanoparticle tracking analysis (NTA) and nano flow cytometry (NanoFCM) (FIG. 1A-D). Transmission electron microscopy (TEM) documented an intact double-membrane nanostructure of PEP vesicles (FIG. 1A). PEP identity was confirmed in different cGMP manufactured lots with the expression of exosomal markers, CD63, CD9 and Alix (FIG. 1B) in tandem with additional release criteria. The hydrodynamic diameter of PEP had a mean value of 129.7 nm (FIG. 1C). Further analysis of PEP by NanoFCM showed a similar mean diameter of 123.49 nm (FIG. 1D). This multi-parametric quality control evaluation helped validate the uniform exosome content of PEP.

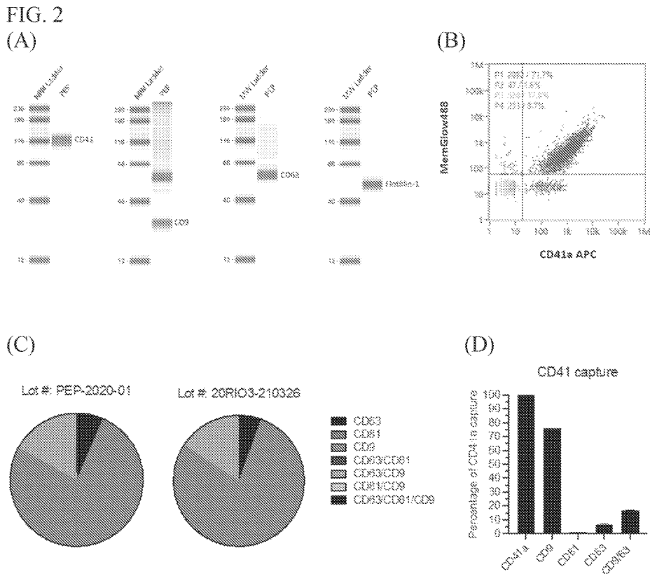

Additional surface marker characterization was conducted (FIG. 2) using automated western blot (JESS, Protein Simple), nano-flow cytometry (Flow Nanoanalyzer, NanoFCM), and affinity-capture based probes (ExoView R200, Nanoview Biosciences). Western Blot analysis demonstrates the presence of exosomal surface markers CD9, CD63, and Flotillin-1 as well as the platelet-specific marker CD41 (FIG. 2A). Nano flow cytometry analysis indicates the lipid membrane bound vesicles (MemGlow 488+) population is also positive for the CD41 platelet specific surface marker, indicating the vesicles are of platelet origin (FIG. 2B). Affinity-based capture of vesicles with subsequent fluorescent antibody staining indicates that the CD41+ captured population of vesicles is enriched in CD9 surface marker with both CD63 and CD81 detected at decreasing levels, respectively (FIG. 2C-D).

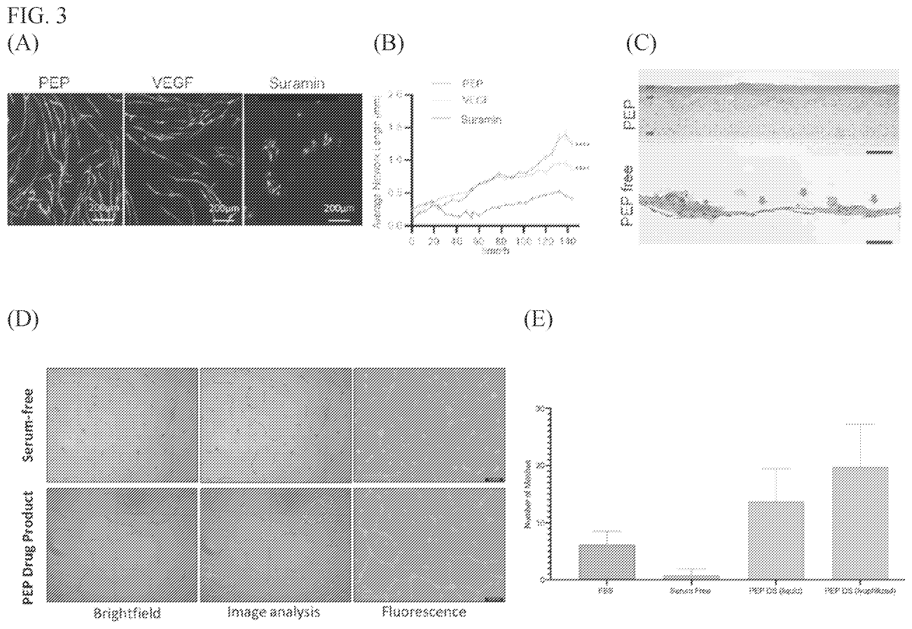

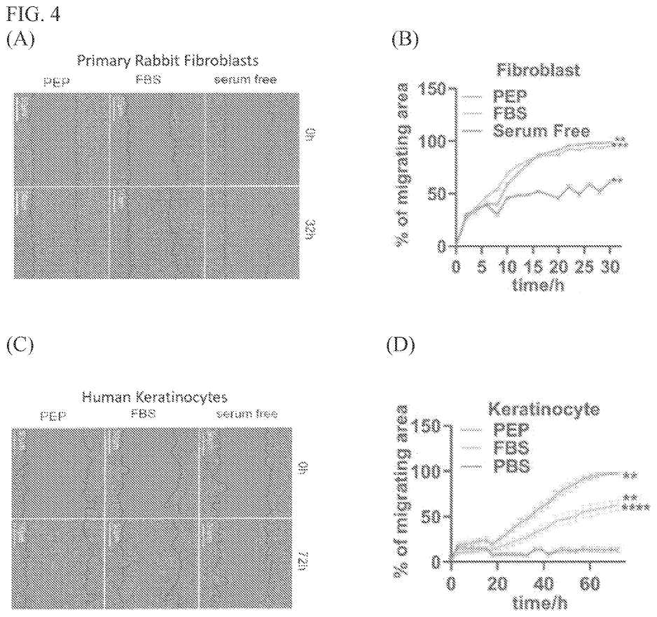

Next, PEP stimulation of skin cell healing was evaluated in vitro (FIG. 3-4) through effects on neovascularization and cell proliferation. Human umbilical vascular endothelial cells (HUVECs) were cultured with PEP, VEGF (vascular endothelial growth factor) or suramin (a VEGF inhibitor) on a fibroblast monolayer. PEP was noted to stimulate angiogenesis in HUVECs more effectively than VEGF, as shown by a marked increase in endothelial tube formation (FIG. 3A-B). Human keratinocytes (hKC) cultured in 3D with PEP showed differentiation of hKC in an air-liquid interface culture and regenerated a normal epidermal architecture within 21 days (FIG. 3C). The angiogenic potential of PEP was further evaluated by culturing HUVECs in an extracellular matrix and treating them with PEP. PEP significantly increased the ability of HUVECs to form tube networks (meshes) over FBS and serum-free controls, indicating that PEP promotes the formation of new vasculature (FIG. 3D-E). PEP furthermore promoted migration of primary rabbit dermal fibroblasts and hKCs as documented in a wound scratch assay (FIG. 4A-D).

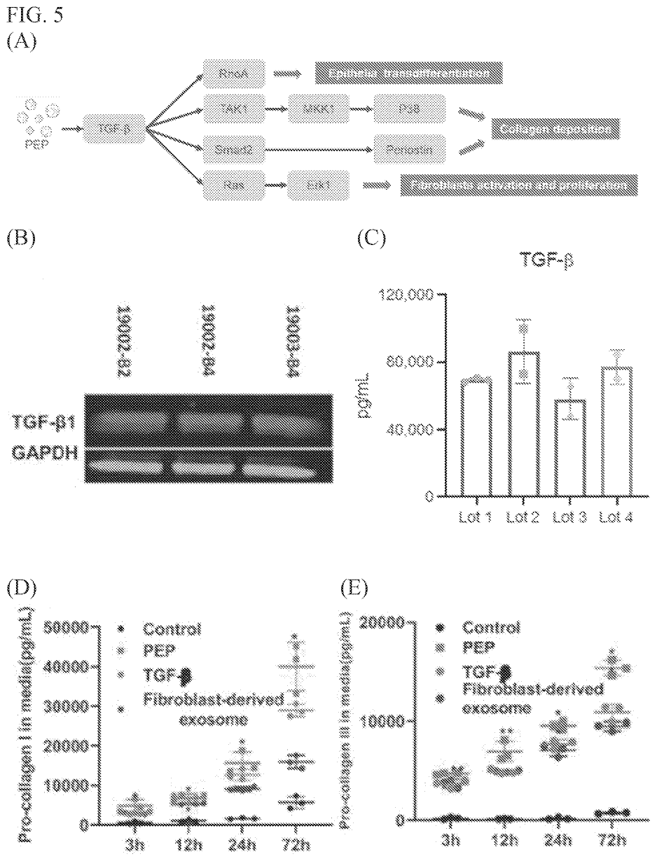

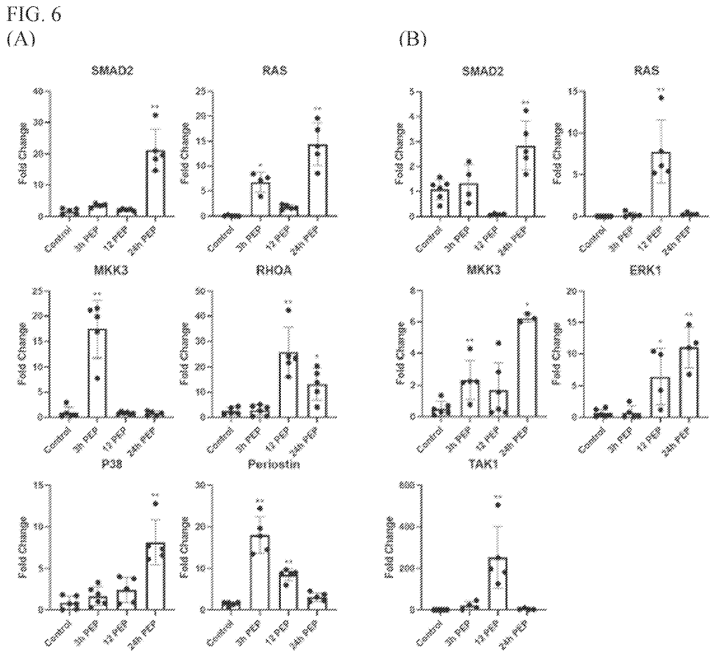

Further studies pin-pointed PEP encapsulated TGF-β as a driver for wound healing events (FIG. 5A). The presence of TGF-β was confirmed via Western Blot (FIG. 5B) and ELISA-based assays (FIG. 5C). Treatment of human fibroblasts (hFB) with PEP significantly augmented collagen I and collagen III secretions versus control (FIG. 5D-E). To confirm TGF-β activity, downstream targets were probed in both hFB and hKC. Compared to control, PEP-treated hKC upregulated TGF-β targets including Smad2, Ras, MKK3 (Mitogen-activated protein kinase kinase 3), RhoA (Ras homolog family member), P38, and periostin, facilitating epithelial transdifferentiation (FIG. 6A). Upregulation was also observed in PEP-treated hFB (FIG. 6B). The increased expression of Smad2, Ras, MKK3, Erk1 (Extracellular signal-regulated kinases), and TAK1 (Transforming growth factor beta-activated kinase 1) confirmed the ability of PEP to donate TGF-β and promote fibroblast activation, proliferation, and collagen deposition in the wound area.

### TISSEEL-PEP Biogel Promotes Ischemic Wound Healing In Vivo

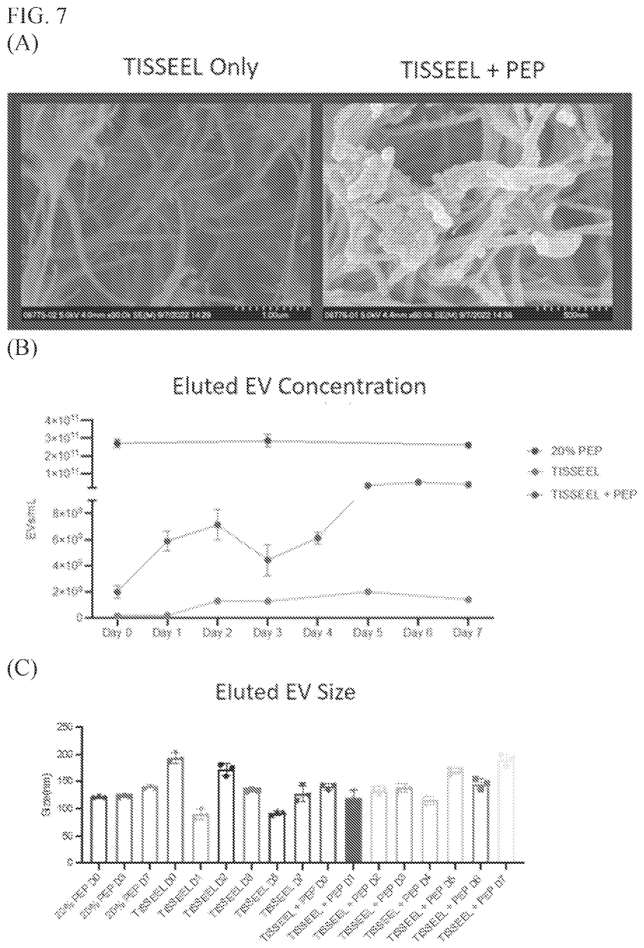

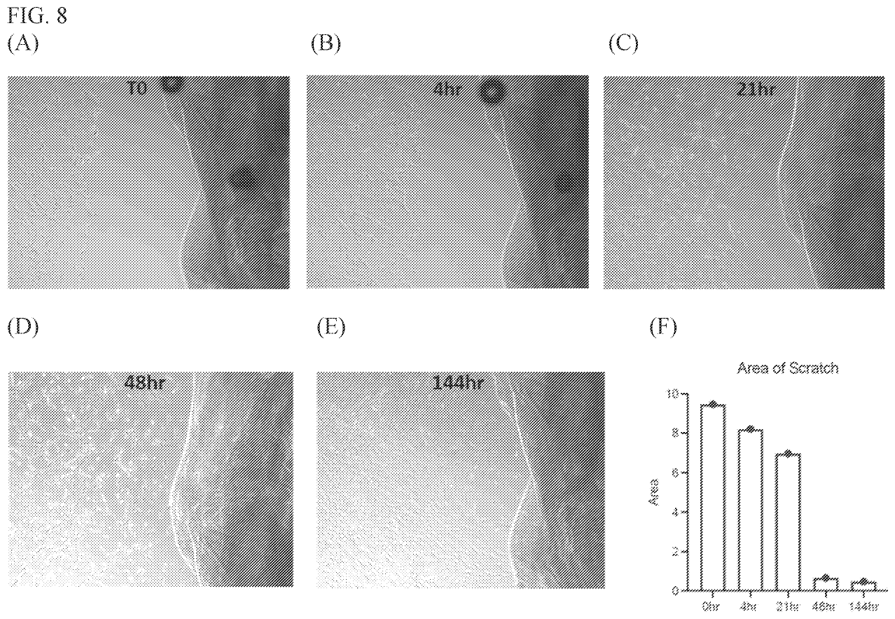

A fibrin sealant (TISSEEL, Baxter International, Inc., Deerfield, IL) was evaluated as a delivery vehicle for PEP to administer the extracellular vesicles to the wound bed (FIG. 7). PEP extracellular vesicles bind to the fibrin fibrils within the fibrin sealant (FIG. 7A), providing sustained release of the vesicles over the course of seven days (FIG. 7B). There was no significant change in vesicle size after mixing with, and elution from, the fibrin sealant (FIG. 7C), indicating that the vesicles remain intact and significant aggregation does not occur. To further evaluate the biocompatibility of the PEP-TISSEEL combination, an in vitro scratch assay was conducted with the PEP-TISSEL mixture administered into the scratch site (FIG. 8A-F). There was rapid progression of the cells towards the biopotentiated fibrin scaffold and closure of the scratch (FIG. 8F).

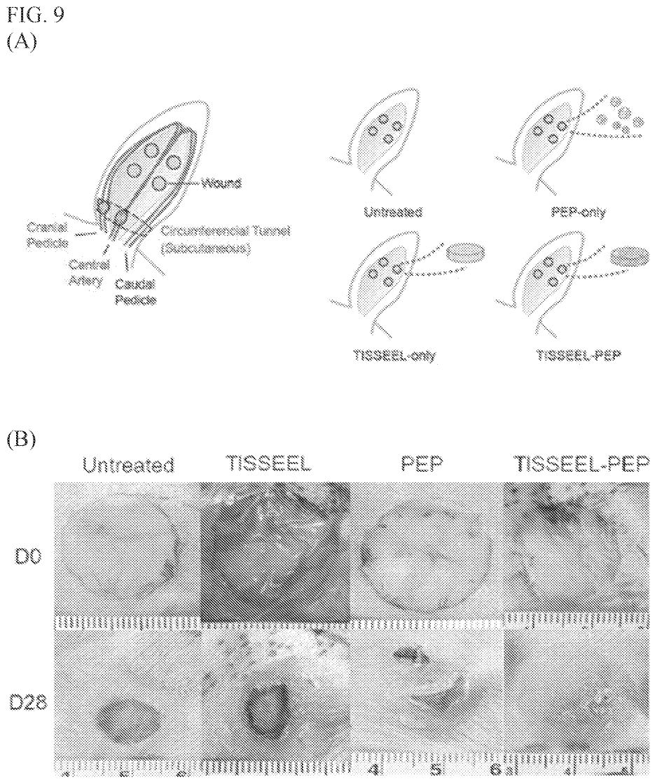

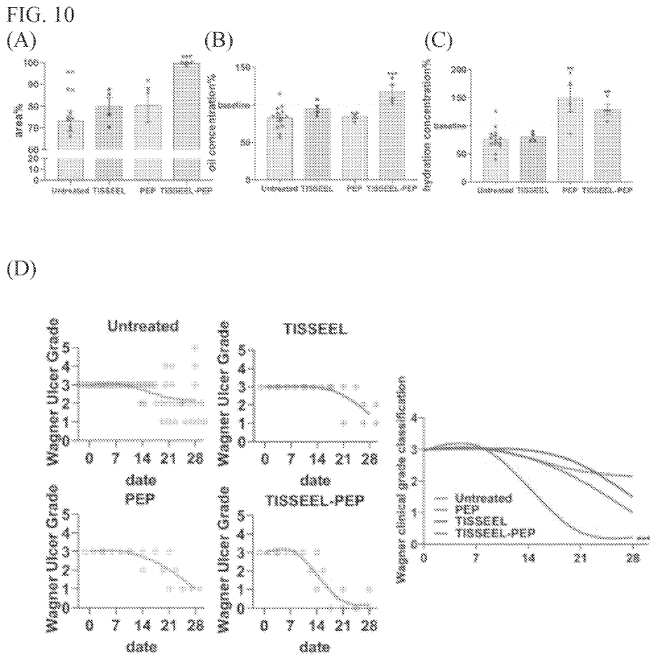

Translating in vitro evidence that PEP can stimulate skin regenerative events, the therapeutic potential of PEP in conjunction with a clinically established wound surgical sealant (TISSEEL, Baxter International, Inc., Deerfield, IL) was investigated. Wound healing in an ischemic rabbit ear model was used to evaluate the efficacy of a PEP-biopotentiated TISSEEL biogel (TISSEEL-PEP) compared with control groups including untreated, TISSEEL-only treated, and PEP-only treated animals (FIG. 9A). At day 28, persistent wounds were found in all groups except the TISSEEL-PEP group (FIG. 9B). TISSEEL-PEP induced the fastest wound closure rate (FIG. 10A).

Further, skin hydration and oil levels are markers of healed skin. Both oil concentration (FIG. 10B) and hydration (FIG. 10C) were significantly higher in TISSEEL-PEP treated animals, suggesting a restoration of cutaneous homeostasis. Results were further supported by Wagner Ulcer classification analysis showing TISSEEL-PEP biogel expedited wound healing (FIG. 10D). Collectively, these findings suggest that TISSEEL-PEP facilitates ischemic wound healing.

### PEP Contributes to Wound Tissue Reorganization

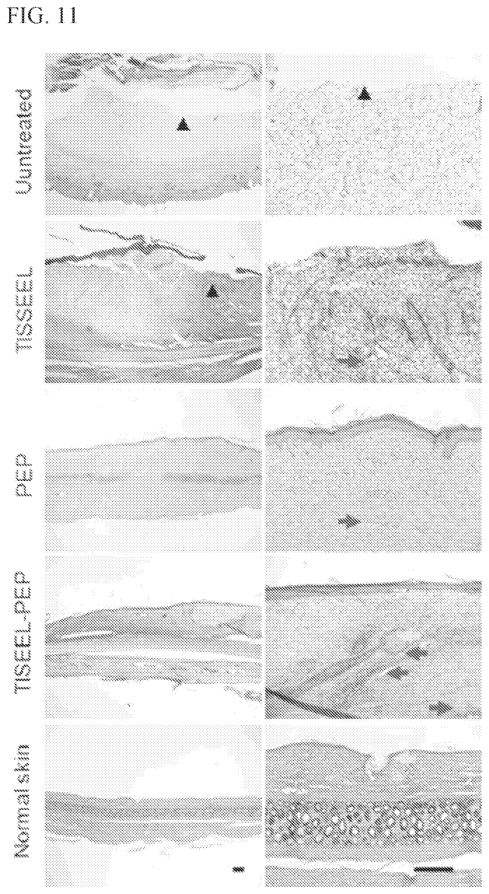

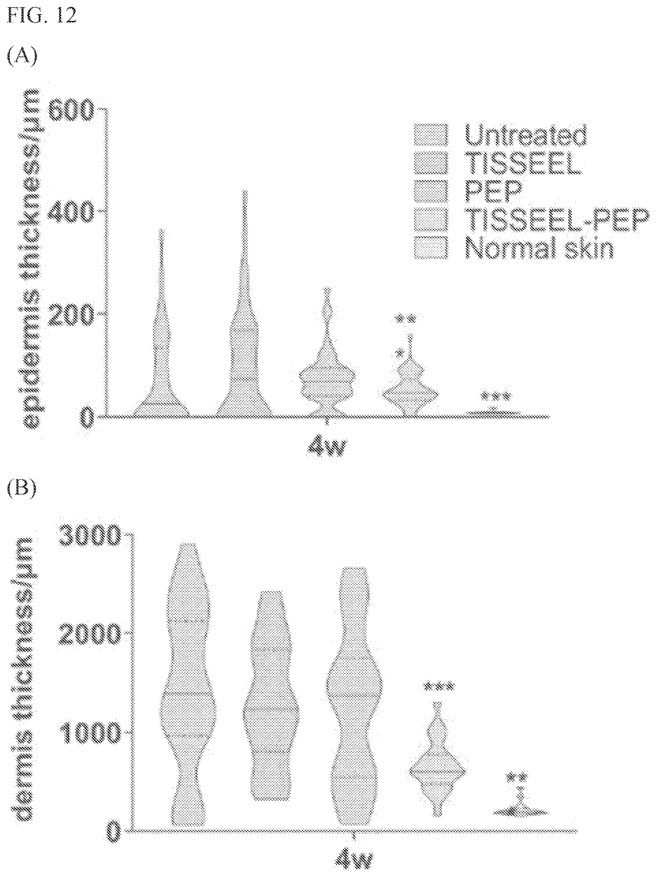

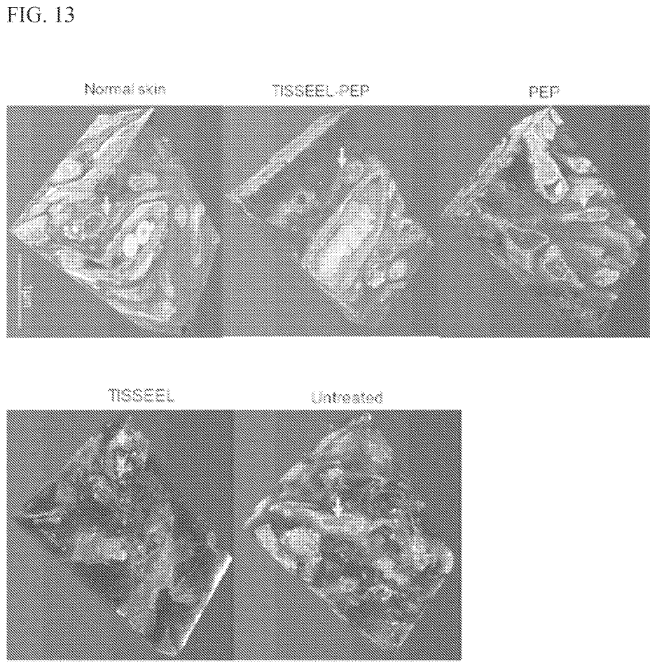

To evaluate the quality of the regenerated skin, physiology of the healed skin was examined. With follow-up up to four weeks, the TISSEEL-PEP treated wounds restored normal dermal architecture comparable to normal skin, while control groups demonstrated abnormal architecture (FIG. 11). One-third of the untreated animals had exposed cartilage and minimal collagen deposition, confirming the severity of the applied model. TISSEEL-PEP wounds also redeveloped hair follicles and sebaceous glands, absent in other groups. Evaluation of skin structure showed that TISSEEL-PEP treated wounds showed superiority in organized epidermal structures with normalization of epidermal thickness (FIG. 12A-B). Accelerated re-epithelization, which minimizes scar formation and prevents trans-epidermal water loss, may be attributed to PEP-stimulated keratinocyte migration and differentiation. To better compare tissue structures, three-dimensional electron microscopy (3D-EM) was used to visualize the full thickness of the wound structure (FIG. 13). Control group tissues showed unorganized and low-density collagen fibers, and PEP-only animals displayed aligned collagen fibers typical of new scar formation. In contrast, TISSEEL-PEP animals exhibited a basket-weave collagen structure, which is similar to a mature extracellular matrix in normal skin (FIG. 13).

### PEP Biogel Drives TGF-β Signaling to Promote Collagen Organization

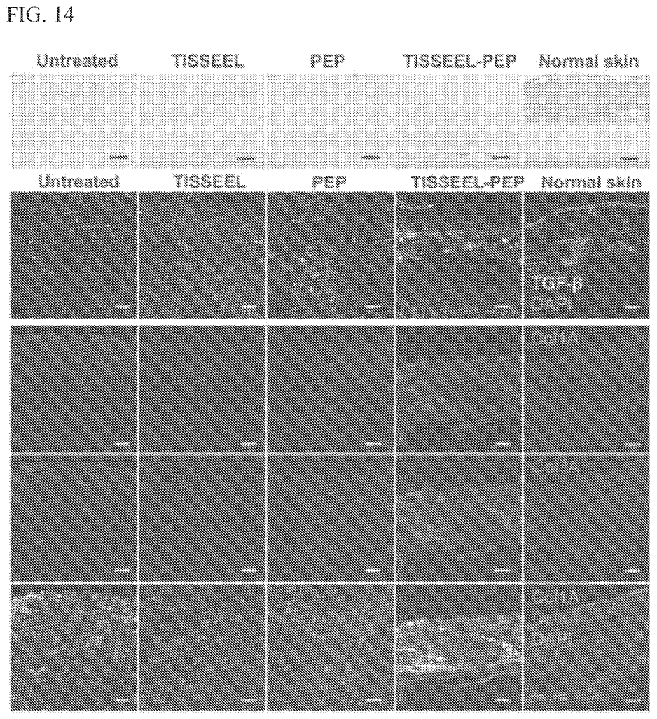

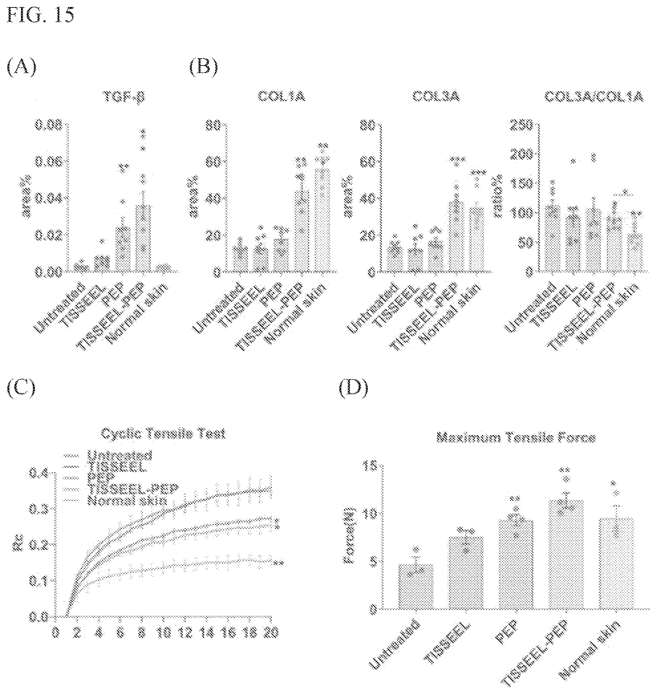

With different collagen organization and expression among treated groups, collagen distribution and TGF-β expression were explored in vivo, probing for re-epithelization, collagen synthesis, and deposition (FIG. 14). Consistent with augmented cell migration (FIG. 4, FIG. 8) and induction of gene expression downstream of TGF-β (FIG. 6) in vitro, TISSEEL-PEP stimulated higher tissue expression of TGF-β (FIG. 15A), driving collagen type I (COL1A) and type III (COL3A) expression (FIG. 15B). While having similar collagen density, the TISSEEL-PEP group had a higher COL3A/COL1A ratio compared to the normal skin group, which may suggest healing with less scar formation as mediated by COL3A. In comparison, the control group showed delayed healing with scar-type cell alignment and abnormal collagen distribution (FIG. 9B).

Since there were different collagen concentrations and organization among groups, collagen content could affect skin biomechanical properties. When wound skin was subjected to cyclic stretching, PEP-only and TISSEEL-PEP tissue behaved the closest to uninjured skin, suggesting functional collagen content versus other treatment groups (FIG. 15C). In contrast, untreated and TISSEEL-only groups showed the poorest elasticity. The tensile strength test demonstrated that TISSEEL-PEP tissue had a similar strength to uninjured skin (FIG. 15D). These findings suggest that in the setting of ischemia, TISSEEL-PEP can regenerate skin, reinstating biomechanical integrity similar to normal skin. TISSEEL-PEP treatment triggers transcriptional remodeling underlying pro-wound healing events.

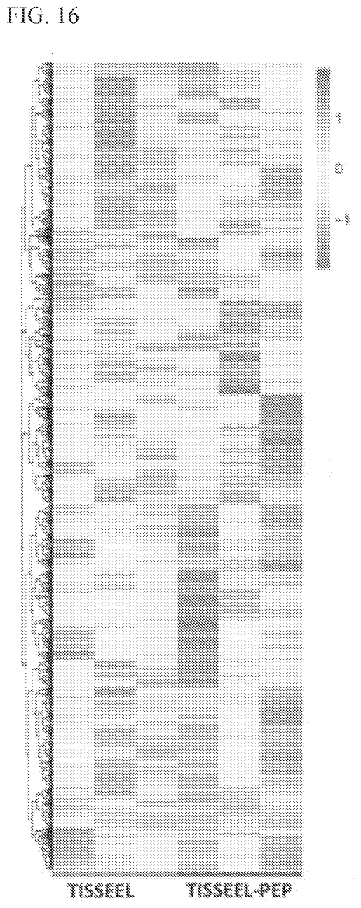

Molecular events underlying TISSEEL-PEP driven regenerative outcomes were probed by transcriptome profiling evaluating over 700 gene targets (FIG. 16). Compared to the TISSEEL group, 213 genes were significantly up-regulated and 523 genes were down-regulated in the PEP-TISSEEL group. Gene ontology enrichment analysis and KEGG pathway analysis showed that the PEP potentiated biogel modulated genes related to pro-wound healing (Table 1).

TABLE 1
Gene ontology enrichment analysis

| Up-regulated | | | Down-regulated | | |
|---|---|---|---|---|---|
| GO Biological process | # genes | p-value | GO Biological process | # genes | p-value |
| Skin development | 35 | 5.45 × 10⁻¹⁷ | Positive regulation of cell | 16 | 3.29 × 10⁻¹⁰ |
| | | | cycle process | | |
| Molting cycle | 16 | 1.29 × 10⁻⁸ | Leukocyte cell-cell | 16 | 1.16 × 10⁻⁸ |
| | | | adhesion | | |
| Hair cell differentiation | 14 | 6.94 × 10⁻⁸ | Positive regulation of | 18 | 4.38 × 10⁻⁸ |
| | | | cytokine production | | |
| Fat cell differentiation | 20 | 1.25 × 10⁻⁷ | T cell proliferation | 12 | 1.59 × 10⁻⁷ |
| Bone development | 19 | 3.59 × 10⁻⁷ | Lymphocyte | 14 | 3.28 × 10⁻⁷ |
| | | | proliferation | | |
| Mammary gland | 304 | 3.99 × 10⁻⁶ | Collagen metabolic process | 8 | 3.67 × 10⁻⁶ |
| epithelium development | | | | | |
| Extracellular structure | 130 | 7.01 × 10⁻⁶ | Neutrophil-mediated | 5 | 6.33 × 10⁻⁶ |
| organization | | | immunity | | |
| Appendage morphogenesis | 14 | 1.70 × 10⁻⁵ | Response to wounding | 15 | 1.82 × 10⁻⁵ |
| Water homeostasis | 7 | 1.46 × 10⁻⁴ | Regulation of NIK/NF- | 7 | 2.19 × 10⁻⁵ |
| | | | κB signaling | | |
| Regulation of | 13 | 1.17 × 10⁻² | Nitric oxide biosynthetic | 6 | 4.12 × 10⁻⁵ |
| angiogenesis | | | process | | |

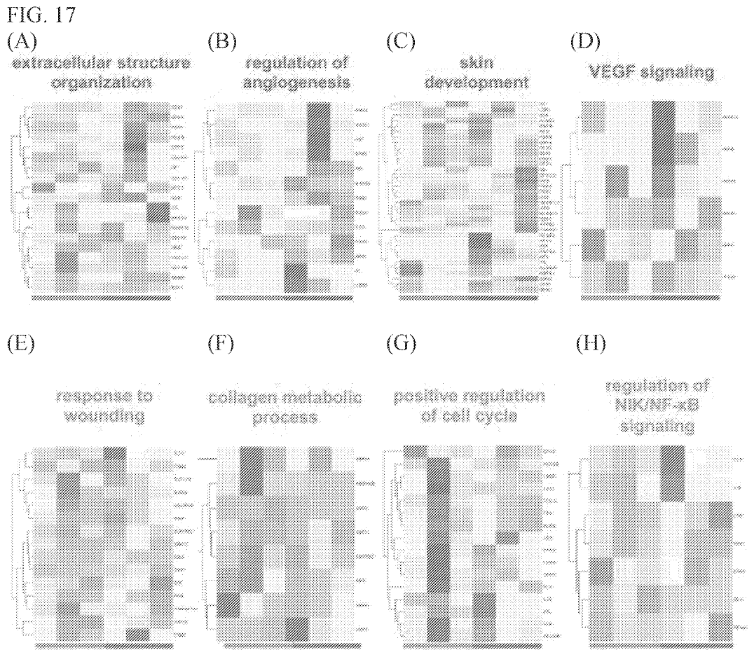

Specifically, genes related to extracellular organization, angiogenesis, skin development, and VEGF signaling were upregulated (FIG. 17A-D). Genes related to the TGF-β pathway and NIK/NF-kB signaling were downregulated (FIG. 17E-H). Further analysis pinpointed PEP effects with regulation of the TGF-β pathway and NIK/NF-KB signaling. These findings are consistent with in vivo results that PEP regulates downstream mediators of TGF-β including increased RhoA, Smad2, TAK1 and Ras pathway (FIG. 6), underpinning enhanced epithelization, fibroblast activation, and collagen production. Interestingly, repressed collagen metabolism was observed in TISSEEL-PEP-treated wounds, which suggests an earlier transition to the remodeling phase of wound healing. This was further supported by the transcriptional and phenotypic development of skin development and maturation.

The results provided here document the therapeutic potential of PEP biopotentiated biogel as a cell-independent, off-the-shelf, regenerative platform for ischemic wound healing. PEP, through donation of bioactive TGF-β, drove mitogenic events in dermal progenitor cells to institute rapid healing in ischemic wounds characterized by epithelial transdifferentiation and enhanced collagen deposition and organization. Furthermore, TISSEEL-PEP-treated wounds demonstrated restored architecture and gene expression profile favoring a physiologic healing process. The concert of biological events driven by TISSEEL-PEP resulted in regenerated tissue that had properties akin to normal skin in histological, biomechanical, and functional assessment.

Global transcriptome fingerprint of PEP-devoid versus PEP-potentiated treatment groups highlighted normalization of molecular events towards a state of health. Taken together, the data presented herein demonstrate that PEP serves as an off-the-shelf regenerative exosome product that engenders a TGF-β-centric program within the ischemic wound bed to promote wound healing.

Evidence of positive effects on multiple biological activities in healing process was observed in this study. In particular, angiogenic events were observed on histology in the TISSEEL-PEP group, suggesting that PEP may also target endothelial cell activity. This was further corroborated in vivo as transcriptome profiling revealed higher expression of genes, including VEGF signaling, related to angiogenesis. Furthermore, tissue transcriptome profiling suggested downregulation of inflammatory and NIK/NF-KB related events suggesting that PEP likely has a polyvalent action in tissues to drive regenerative events.

In summary, the data presented herein show a specialized PEP biopotentiated hydrogel promoted ischemic wound healing through regulating epithelial transdifferentiation, collagen reorganization, and overall guiding skin tissue development via the TGF-β pathway. Thus, PEP offers a cell-free regenerative therapy with promising therapeutic potential for patients with chronic ischemic wounds. PEP preparations can therefore serve as a technical platform, providing an off-the-shelf, cell-independent regenerative therapy.

In one aspect, this disclosure describes compositions and methods for treating a wound in a subject. In various embodiments, the wound may be an ischemic wound (e.g., an ischemic ulcer), a puncture wound, a laceration, an abrasion, a surgical wound, a skin graft, or a traumatic wound. Generally, the compositions include a PEP preparation and a pharmaceutically acceptable carrier. In one or more embodiments, the pharmaceutically acceptable carrier can include, for example, a surgical glue or a tissue adhesive.

As used herein, a “subject” can be a human or any non-human animal. Exemplary non-human animal subjects include, but are not limited to, a livestock animal, a companion animal, or a laboratory animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

Thus, the method includes administering an effective amount of the composition to a wound in need of repair. An “effective amount” is an amount effective to reduce the time to wound closure compared to a suitable comparable wound that is either untreated or receives different wound closure treatment. In one or more embodiments, an “effective amount” is an amount effective to increase angiogenesis, increase migration of fibroblasts into the wound, or increase migration of keratinocytes into the wound compared to a comparable untreated wound. In one or more other embodiments, an “effective amount” is an amount effective to increase TGF-β, COL IA, or COL3A compared to a comparable untreated wound. In one or more other embodiments, an “effective amount” is an amount effective to decrease Wagner Ulcer Classification grade of the wound compared to a comparable untreated wound. In one or more other embodiments, an “effective amount” is an amount effective to decrease reaction force variation (Re) or increase resistance to tensile force compared to a comparable untreated wound. In one or more other embodiments, an “effective amount” is an amount effective to increase expression of SMAD2, RAS, MKK3, RHOA, P38, or periostin in keratinocytes compared to untreated keratinocytes. In one or more other embodiments, an “effective amount” is an amount effective to increase expression of SMAD2, RAS, MKK3, ERK.I, or TAK1 in fibroblasts compared to untreated fibroblasts. In certain embodiments, the comparative control can be a wound, keratinocytes, or fibroblasts treated with a tissue sealant or surgical glue without PEP. In certain embodiments, the comparative control can be a wound, keratinocytes, or fibroblasts treated with PEP in the absence of a tissue sealant or a surgical glue.

PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. In one or more embodiments, the pharmaceutically acceptable carrier can include a hydrogel. In certain embodiments, the pharmaceutically acceptable carrier can include a fibrin sealant (e.g., TISSEEL, Baxter International, Inc., Deerfield, IL; VISTASEAL, Johnson & Johnson Corp., New Brunswick, NJ; EVICEL, Johnson & Johnson Corp., New Brunswick, NJ; ARTISS, Baxter International, Inc., Deerfield, IL; TACHOSIL, Corza Health, Inc., Del Mar, CA; RECOTHROM, Baxter International, Inc., Deerfield, IL), tissue sealant, or surgical glue. Use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. So, for example, a pharmaceutically acceptable carrier can include a hydrogel that includes a basement membrane protein (e.g., collagen). Additionally, multiple pharmaceutically acceptable carriers can be combined. Thus, in certain embodiments, the pharmaceutically acceptable carrier can include a hydrogel that includes, for example, a thrombin sealant, fibrin sealant, tissue sealant, or surgical glue. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained.

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to nervous tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from about 1×10⁶ PEP exosomes to about 1×10¹⁵ PEP exosomes to the subject, although in one or more embodiments the methods may be performed by administering PEP in a dose outside this range. In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, at least 2×10¹² PEP exosomes, at least 3×10¹² PEP exosomes, at least 4×10¹² PEP exosomes, at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any a minimum dose identified above and any maximum dose that is greater than the selected minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

Alternatively, the method can include administering sufficient PEP to provide a dose of, for example, from about a 0.01% solution to a 100% solution to the subject, although in one or more embodiments the methods may be performed by administering PEP in a dose outside this range. As used herein, a 100% solution of PEP refers to approximately 75 mg of PEP solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the selected minimum dose. For example, in one or more embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose may be administered as a single dose, continuously over 24 hours, or as two administrations, which may be equal or inequal. When multiple administrations are used to deliver a single dose, the interval between administrations may be the same or different. In certain embodiments, PEP may be administered from a one-time administration, for example, during a surgical procedure.

In one exemplary embodiment, the PEP composition may be administered as soon as a subject presents with a wound in need of repair. The subject may receive a single administration of the PEP composition or may receive multiple administrations of the PEP composition. In certain embodiments in which multiple administrations of the PEP composition are administered to the subject, the PEP composition may be administered as needed until the wound is healed to satisfaction. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP at an interval (for two administrations) or intervals (for more than two administrations) characterized by a range having endpoints defined by any a minimum interval identified above and any maximum interval that is greater than the selected minimum interval. For example, in one or more embodiments, the method can include multiple administrations of PEP at an interval or intervals of from one day to six months such as, for example, from three days to ten days. In certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique wound healing profile—e.g., protein composition and/or gene expression. In this way, the PEP composition can provide a broader spectrum of wound healing activity than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably to mean one or more than one; and the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The words “preferred” and “preferably” refer to embodiments of the invention that may afford certain benefits under certain circumstances. However, other embodiments may also be preferred under the same or other circumstances. Furthermore, the description of one or more preferred embodiments does not imply that other embodiments are not useful and is not intended to exclude other embodiments from the scope of the invention.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Examples

### PEP Preparation

Clinical grade Purified Exosome Product (Rion LLC, Rochester, MN) vials were acquired from the Advanced Product Incubator biomanufacturing facility at Mayo Clinic. Briefly, PEP represents a highly regenerative exosome fraction that is isolated from apheresis platelets through a serial filtration, enucleation, and centrifugation steps. An ultimate encapsulation step allows lyophilization of the exosome population retaining the integrity of the exosomal lipid bilayer. Additional process details provided in U.S. Pat. No. 10,596,123 and International Publication No. WO 2019/118817 A1. A vial of sealed PEP (representing 5×10¹² exosomes) was dissolved in 1 mL phosphate buffered saline (PBS), which was defined as a 100% (w/v). Before using, the PEP solution was filtered through a STERIFLIP-GP sterile 0.22-μm filter system (MilliporeSigma, Burlington, MA). The 100% PEP solution was diluted in PBS for characterization or dissolved in culture medium for cell culture.

### Electron Microscopy

Suspended PEP was dropped on formvar carbon-coated nickel grids for electro-microscopic verification. After staining with 2% uranyl acetate, the grids were air-dried and visualized by transmission electron microscope (TEM, H-7650 Hitachi High-Technology Science Corp., Tokyo, Japan).

### Exosome Characterization: NANOSIGHT, NanoView, and NanoAnalyzer

NANOSIGHT NS300 (Malvern Panalytical Ltd., Malvern, UK) was used for real-time characterization of the PEP vesicle size and concentration.

NanoAnalyzer (NanoFCM) was used for nano flow cytometry real-time characterization of PEP vesicle size and concentration. Furthermore, PEP was fluorescently labeled with MEMGLOW488 lipid membrane stain and CD41a APC antibody (platelet-specific surface marker).

NanoView: Two lots of PEP were reconstituted and further diluted 1000-fold. Fifty microliters of sample were incubated on CD41a capture chip for 16 hours. Chips were washed and incubated with antibodies for CD9, CD63, and/or CD81 for fluorescent labeling of PEP expressing the aforementioned surface markers. Data was collected using an R100 reader and analyzed with EXOVIEW Scanner 3.0 software (NanoView Biosciences, Inc., Brighton, MA).

### Western Blot Analysis

PEP vesicles were reconstituted in RIPA-based lysis buffer and homogenized with an ultrasonic homogenizer (Branson Ultrasonics, Brookfield, CT). Protein concentration was quantified using a BCA protein assay kit (Thermo Fisher Scientific, Inc., Waltham, MA). Equal amounts of protein were dissolved with SDS-PAGE gel and probed on to ODYSSEY nitrocellulose membranes (LI-COR Biosciences, Inc., Lincoln, NE). Overnight incubation was with diluted antibodies against CD63 (1:1000, Abcam ab59479, Abcam plc, Cambridge, UK), CD9 (1:1000, Cell Signaling 13174s, Cell Signaling Technology, Inc., Danvers, MA), Alix (1:1000, Cell Signaling 2171s, Cell Signaling Technology, Inc., Danvers, MA), GAPDH (1:1000, Cell Signaling 2118s, Cell Signaling Technology, Inc., Danvers, MA), which was followed by appropriate diluted secondary antibody (Invitrogen, Carlsbad, CA). Bound antibody was detected using the Odyssey System (LI-COR Biosciences, Inc., Lincoln, NE).

### Simple Western Blot Analysis

PEP vesicles were concentrated using the EXOEASY Maxi kit (QIAGEN, Hilden, Germany). Total protein concentration was quantified via BCA protein assay kit (Thermo Fisher Scientific, Inc., Waltham, MA). The JESS automated Western blot system (ProteinSimple, Santa Clara, CA) was used according to the manufacturer's protocol. Protein was loaded at 1 mg/mL to detect CD9 and Flotillin-1, at 0.5 mg/mL to detect CD63, and at 0.02 mg/mL to detect CD41. Primary antibodies used include rabbit anti-human CD9 (1:30, Cell Signaling 13403S, Cell Signaling Technology, Inc., Danvers, MA), rabbit anti-human Flotillin-1 (1:50, Abcam ab133497, Abcam plc, Cambridge, UK), rabbit anti-human CD63 (100 μg/mL, MAB50482, R&D Systems, Inc., Minneapolis, MN), and rabbit anti-human CD41 (1:30, NBP1-84581, Novus Biologicals, LLC, Centennial, CO). Data was analyzed on COMPASS software (ProteinSimple, Santa Clara, CA).

### Automated ELISA by Ella, ProteinSimple

PEP vesicles were reconstituted in 1×RIPA lysis buffer, vortexed, and incubated for five minutes at room temperature. Samples were centrifuged at 14,000 rpm for 10 minutes, then supernatants were filtered through a 0.22 μm surfactant-free cellulose acetate (SFCA) filter syringe. Latent TGF-β was activated to the immunoreactive form with 1 N HCl, then neutralized with 1.2 N NaOH/0.5 M HEPES. Samples were diluted with sample diluent. Samples were loaded onto the TGF-β cartridge and run on an automated ELISA instrument (ELLA, ProteinSimple, Santa Clara, CA).

Quantitative Real-Time Polymerase Chain Reaction (qRT-PCR)

The PEP-stimulated gene expressions of human keratinocytes (ABC-TC536S, AcceGen, Fairfield, NJ) and fibroblasts (C0135C, Gibco, Thermo Fisher Scientific, Inc., Waltham, MA) were tested by qRT-PCR. The keratinocytes and fibroblasts were treated with 5% PEP for three hours, 12 hours, or 24 hours, while blank DMEM served as a control. Total RNA was isolated from keratinocytes and fibroblasts using TRI REAGENT (Molecular Research Center, Inc., Cincinnati, OH) in accordance with the manufacturer's standard protocol. Complementary DNA (cDNA) was reverse-transcribed from equal amounts of RNA (1 μg) by iSCRIPT cDNA Synthesis Kit (Bio-Rad laboratories, Inc., Hercules, CA). All runs were performed using SYBR Green PCR Master Mix (Quantabio, Beverly, MA) on a thermocycler (C1000 TOUCH, Bio-Rad Laboratories, Inc., Hercules, CA) for the following genes: Smad2, Ras, MKK3, Erk1, Periostin, P38, RhoA, and TAK1. Glyceraldehyde-3-phosphate dehydrogenase (GAPDH) was selected as an internal control. To control for pipetting errors, each cDNA sample was run in duplicate. The primers used in the amplification are listed in Table 2. Data from target genes was normalized to GAPDH and then calculated using the 2^{-ΔCt} method.

TABLE 2
PCR primer sequences

| | Forward | Reverse |
|---|---|---|
| GAPDH | GAGTCAACGG | TTGATTTTGG |
| | ATTTGGTCGT | AGGGATCTCG |
| | (SEQ ID NO: 1) | (SEQ ID NO: 2) |
| H-Ras | ACGACGATGA | ATGGCGCTGT |
| | CAAGACGGAA | ACTCCTCCT |
| | (SEQ ID NO: 3) | (SEQ ID NO: 4) |
| Smad2 | ACTAACTTCC | GTTGGTCACT |
| | CAGCAGGAAT | TGTTTCTCCA |
| | (SEQ ID NO: 5) | (SEQ ID NO: 6) |
| Erk1 | CCTGCGACCT | CAGGGAAGAT |
| | TAAGATTTGT | GGGCCGGTTA |
| | GATT | GAGA |
| | (SEQ ID NO: 7) | (SEQ ID NO: 8) |
| TIMP-1 | TGACATCCGG | TGCAGTTTTC |
| | TTCGTCTACA | CAGCAATGAG |
| | (SEQ ID NO: 9) | (SEQ ID NO: 10) |
| Periostin | ATGATTCCCT | GAAGGAATAA |
| | TTTTACCCAT | TCATGCCATT |
| | GTTTTCTCTA | TTTTAAGTCC |
| | (SEQ ID NO: 11) | (SEQ ID NO: 12) |
| MKK3 | CTTGGTGACC | CTTCTGCTCC |
| | ATCTCAGAAC | TGTGAGTTCA |
| | TGG | CG |
| | (SEQ ID NO: 13) | (SEQ ID NO: 14) |
| P38 | CCAATGCCTA | TGGGAAGTGA |
| | CGACAAGACA | CCTCGTTTGC |
| | GC | CA |
| | (SEQ ID NO: 15) | (SEQ ID NO: 16) |
| Nf-kb | GCAGCACTAC | TCTGCTCCTG |
| | TTCTTGACCA | AGCATTGACG |
| | CC | TC |
| | (SEQ ID NO: 17) | (SEQ ID NO: 18) |
| hRHOA | CGCTTTTGGG | TTGCAGCAAG |
| | TACATGGAGT | GTTTCACAAG |
| | (SEQ ID NO: 19) | (SEQ ID NO: 20) |
| Akt | TGGACTACCT | GTGCCGCAAA |
| | GCACTCGGAG | AGGTCTTCAT |
| | AA | GG |
| | (SEQ ID NO: 21) | (SEQ ID NO: 22) |
| TAK1 | CAGAGCAACT | CATTTGTGGC |
| | CTGCCACCAG | AGGAACTTGC |
| | TA | TCC |
| | (SEQ ID NO: 23) | (SEQ ID NO: 24) |
| NOS2 | GTGGCAGGAC | CATCAGCACA |
| | ATGAAGAAGA | GAGGCAAAGA |
| | A | (SEQ ID NO: 26) |
| | (SEQ ID NO: 25) | |
| TNF-a | CTCATCTACT | GTTGACCTTG |
| | CCCAGGTTCT | TTCGGGTAGG |
| | CT | (SEQ ID NO: 28) |
| | (SEQ ID NO: 27) | |
| IL-6 | GTCAACTGCA | AGCAGGCAGG |
| | TGAACAGAAA | TCTCATTATT |
| | GG | C |
| | (SEQ ID NO: 29) | (SEQ ID NO: 30) |
| IL-1b | CGAACCCAAG | TGGAAAGTGT |
| | CTACAGGAAT | GTGTCCAATC |
| | AG | A |
| | (SEQ ID NO: 31) | (SEQ ID NO: 32) |
| IL-10 | CCTGTGGGAT | GCTCGGCTTA |
| | TTGAGTGTCT | GGAGTTAGAA |
| | TA | AG |
| | (SEQ ID NO: 33) | (SEQ ID NO: 34) |
| CD206 | GGTGACATCC | CCAGGCATAG |
| | ACGACTACTT | CTGTTGTACT |
| | TAG | T |
| | (SEQ ID NO: 35) | (SEQ ID NO: 36) |

### Type I/III Collagen ELISA

Collagen I and Collagen III concentrations were measured separately by ELISA (R&D Systems, Inc., Minneapolis, MN). The absorbance at 450 nm was measured using a microplate spectrophotometer (FLUOstar Omega, BMG Labtech, Ortenberg, Germany).

### HUVEC Angiogenesis Assay for Co-Culture

INCUCYTE 96-well angiogenesis assay (Essen BioScience, Inc., Ann Arbor, MI) was performed according to the manufacturer's protocol. Briefly, lentivirus infected green fluorescent protein (GFP) expressing HUVECs (Essen BioScience, Inc., Ann Arbor, MI) were co-cultured with normal human dermal fibroblasts (Essen BioScience, Inc., Ann Arbor, MI) in a 96-well microplate. The plate was placed in an INCUCYTE imager (Essen Bioscience, inc., Ann Arbor, MI) and images were automatically acquired in both phase and fluorescence every six hours for 10 days. At Day 4, small-molecule inhibitors were added on the endothelial tube networks and kept throughout the experiment. The Angiogenesis Analysis Module (Essen BioScience, Inc., Ann Arbor, MI) was used to quantify tube length and branch points. Eight biological replicates were included for each condition.

### HUVEC Angiogenesis Assay in Extracellular Matrix

Angiogenesis kit (Abcam plc, Cambridge, UK) was used to coat each well in a 96-well plate with 50 μl of the extracellular matrix solution while maintaining the plate and reagents on ice. Control wells for each sample tested were included. The plate was transferred to the incubator at 37° C. for one hour to allow for the extracellular matrix solution to form a gel. The PEP samples were diluted with serum-free media with heparin (1 U/mL) for a final concentration of 20% (v/v). HUVECs were added to each well at a density of 3,200 cells per well in 100 μl of serum-free tissue culture media containing heparin at a concentration of 1 U/mL. The plate was placed in the incubator at 37° C. for 18 hours, with images acquired using an INCUCYTE scanner (Essen Bioscience, inc., Ann Arbor, MI) at 10× magnification every three hours. After 18 hours, cell were stained according to the manufacturer's protocol in the angiogenesis assay kit. Briefly, the stain provided in the kit was diluted with wash buffer. Brightfield and fluorescence microscopy images were acquired after cells were stained. Images were uploaded to ImageJ software and analyzed using the Angiogenesis Analyzer tool (Carpentier, et al., Sci Rep 10(1):11568, 2020).

### Cell Migration Assay

Primary rabbit dermal fibroblasts (FB) were isolated from healthy rabbit (around six months old with a body weight of 2.0-3.5 kg) ear skin and maintained in DMEM with 10% fetal bovine serum. Primary human keratinocytes (KC) were purchased from Gibco (C0055C, Thermo Fisher Scientific, Inc. Waltham, MA) and maintained. Cell migration was analyzed by scratch wound assay, which was measured by a live cell imaging system (INCUCYTE S3, Essen BioScience Inc., Ann Arbor, MI). FBs or KCs were seeded in a 96-well plate (Corning, Inc., Corning, NY). Cells were allowed to grow in standard tissue culture condition until confluent, followed by scratching on cell monolayer using a 96-pin WOUNDMAKER device (Essen BiosScience Inc., Ann Arbor, MI). After two phosphate-buffered saline washes and addition of 10% PEP with blank medium, the plate was placed into an INCUCYTE imager (Essen BiosScience Inc., Ann Arbor, MI) for timed imaging.

Adipose-derived MSCs were plated and allowed to grow until 80% confluent. Migration was measured by a scratch wound assay. A wound was created by scratching the cell monolayer using a pipette tip. Cells were washed with PBS and effects of PEP in TISSEEL biogel on cell migration was evaluated by applying a thin line of the biogel into the middle of the scratch created in the monolayer of MSCs. Cells were left to migrate for seven days while continuous imaging was carried out via the INCUCYTE and brightfield microscopy. Scratch area was quantified using ImageJ.

### Skin Tissue Organoid Assay

The skin tissue organoid assay (Thermo Fisher Scientific, Inc., Waltham, MA) was performed according to the manufacturer's protocol. Briefly, human adult epidermal cells (C0055C, Gibco, Thermo Fisher Scientific, Inc., Waltham, MA) were seeded as 750,000 cells/cm² in pre-coated cell culture inserts and cultured with 50 μL EPILIFE growth medium with supplements (Gibco, Thermo Fisher Scientific, Inc., Waltham, MA). After incubating for two days at 37° C. and 5% CO₂, the inserts were repositioned to the desired hanging height in the 24-well plate and the medium was changed while the upper compartment, inside of the cell culture insert, was left empty. Skin tissue inserts were allowed to grow for 28 days post-seeding and then fixed using an overnight incubation in 4% paraformaldehyde at 4° C. Inserts were paraffin embedded and sectioned, followed by processing for hematoxylin and eosin (H&E) staining. Tissue sections were photographed using an Olympus BH-2 microscope (Olympus Life Science, PA) at 400× magnification to examine the stratification of cell layers.

### PEP Biogel Preparation and SEM

To prepare PEP with TISSEEL (Baxter International, Inc., Deerfield, IL), a 2 ml standard kit was used. PEP was dissolved in the fibrinolysis inhibitor solution (human fibrinogen, aprotinin, human albumin, L-histidine, niacinamide, natriumcitrat dihydrate, polysorbate, water) in the TISSEEL fibrin glue preparation kit. The solution was then prepared following standard TISSEEL preparation protocol per manufacturer and mixed with thrombin solution (500 IE/ml)/CaCl₂) (40 μmol/ml) for local administration.

For SEM, samples were fixed in Trump's fixative at 4° C. overnight, washed with PBS and then in water, dehydrated to get dried. Samples were imaged in a cold field emission scanning electron microscope (S-4700, Hitachi High-Tech Global, Tokyo, Japan).

### In Vivo Wound Healing Animal Model

Twelve Female New Zealand white rabbits (around six months old with a body weight of 2.0-3.5 kg) were used for this study. Rabbits were assigned into each treatment group in sequence. Ischemia was induced in both ears by ligating two of the three vascular bundles of the ear. A circular full-thickness skin defect measuring 2 cm in diameter was created on each ear. Ear ischemia after surgery was confirmed using indocyanine green angiography with the SPY Elite florescence imaging system (Stryker Corp., Kalamazoo, MI). Rabbits were then randomly assigned into three groups. In each group, one ear was left as untreated while another side was treated with 0.6 mL TISSEEL (Baxter healthcare Corp., Deerfield, IL), 0.6 mL 20% PEP, or 0.6 mL TISSEEL-PEP (20%) combination. Wound healing was observed and recorded every day for the first week and weekly thereafter until rabbits were sacrificed after four weeks.

### Clinical Assessment of Wound Closure

To grade the wound healing from a clinical perspective, all the ischemia wounds were assessed once a week by board-certified plastic surgeons at Mayo Clinic according to the Wagner Ulcer Grade Classification System.

### Measurement of Wound Hydration Level and Oil Level

Skin hydration and oil level were objectively evaluated using a digital skin moisture/oil detector (Zinnor, Korea). All measurements were taken under standard climate conditions (temperature, 25±1° C.; relative humidity, 50±5 percent).

### Biomechanical Test

Specimens of the full thick wound were cut into 2-mm wide strips. The specimens were then mounted to a tensile testing device using super glue (Gorilla Glue Co., Sharonville, OH). The glue was applied to the bare skin on the ends to prevent slipping from occurring at the site where the specimens were clamped with grips. The grips had a serrated internal surface to reduce slipping. A custom tensile testing machine, with a 25-lb. load cell (MLP-25, Transducer Techniques LLC, Temecula, CA), was used to perform cyclic tensile testing of the specimens. Specimens were tested at a constant strain rate of 0.1 mm/s, with a peak displacement of 1 mm for 20 cycles. A preload of 1 N was applied before starting each test. After the 20^{th} cycle, the specimens were tested to failure with a strain rate of 0.1 mm/s. The motor control and data acquisition were handled via a custom NI LabVIEW 2018 application (National Instruments Corp., Austin, TX), with the load and displacement data sampled at a rate of 50 Hz.

### Histology

Rabbits were sacrificed by four weeks post-surgery. Ear skin from the original site of injury was removed and fixed in 10% neutral formalin overnight at 4° C., then rinsed 24 hours at 4° C. in PBS containing 30% sucrose and 0.1% sodium azide, with PBS changes after approximately 12 hours to eliminate any formalin remnants. Specimens were then embedded in paraffin wax (Thermo Fisher Scientific, Inc., Waltham, MA) by a tissue processor (EXCELSIOR AS, Thermo Fisher Scientific, Inc., Waltham, MA). The specimens were then sliced into longitudinal sections (5 μm) and prepared onto slides (SUPERFROST PLUS, New Erie Scientific LLC, Fremont, CA). After embedding, the specimens were transversally sectioned in 5 μm for further use. Hematoxylin and eosin (H&E) staining and Masson's trichrome staining were performed according to standard procedures.

### Immunohistochemistry IHC Analysis (TGF-β, Col I & III, α-Smooth Muscle Actin, CD31)

Skin sections underwent deparaffinization and were permeabilized with 0.5% Triton X-100 in PBS for five minutes. They were next incubated with blocking buffer (5% normal donkey serum, 0.2% Triton-X in PBS) prior to primary antibody incubation at 4° C. overnight with the following antibodies diluted in blocking buffer: anti-TGF-β (1:400, MAB240-SP, R&D Systems, Inc., Minneapolis, MN), anti-collagen I (1:200, ab24821, Abcam, Cambridge, UK), and anti-collagen III (1:400, ab6310, Abcam, Cambridge, UK). After three 30-minute washes with PBS+0.05% Triton-X, samples were stained for one hour at room temperature with fluorescent secondary antibodies (Thermo Fisher Scientific, Inc., Waltham, MA) followed by two washes with PBS. The slides were mounted with antifade mounting medium with DAPI (h-1200, Vector Laboratories, Inc., Burlingame, CA) and viewed under a spinning-disc confocal microscope (Carl Zeiss Microscopy GmbH, Jena, Germany).

### 3D-EM Reconstruction of Wound Tissue

Full thickness wound samples were fixed, stained, and prepared for serial block-face microscopy using a protocol adapted from that previously described (Hua et al., 2015, Nat Commun 6(1):7923). Briefly, tissue samples were fixed by immersion in 2% glutaraldehyde+2% paraformaldehyde in 0.15 M cacodylate buffer containing 2 mM calcium chloride until further processed (minimum of 24 hours). Fixed samples were washed in 0.15 M cacodylate buffer and incubated at room temperature in 2% osmium tetroxide in 0.15 M cacodylate for 1.5 hours. Without rinsing, samples were incubated in 2.5% potassium ferricyanide+2% osmium tetroxide in 0.15 M cacodylate for another 1.5 hours at room temperature. Following a rinse in deionized water (dH₂O), samples were incubated in 1% thiocarbohydrazide in H₂O for 45 minutes at 50° C. After another rinse in dH₂O, samples were incubated sequentially in 2% osmium tetroxide in H₂O for 1.5 hours at room temperature, 1% aqueous uranyl acetate overnight at 4° C., and 7% lead aspartate solution one hour at 50° C., with several rinses in dH₂O between each reagent.

Following dehydration through a series of ethanol and acetone, samples were infiltrated and eventually embedded in polyepoxide resin (DURCUPAN, MilliporeSigma, St. Louis, MO) and polymerized in a 60° C. oven for a minimum of 24 hours. To prepare embedded samples for placement into the scanning electron microscope and subsequent imaging, 1 mm³ pieces were roughly trimmed of any excess resin and mounted to 8-mm aluminum stubs using silver epoxy EPO-TEK (Epoxy Technology, Inc., Billerica, MA). The mounted sample was then carefully trimmed to a 0.5 mm×0.5 mm×1 mm tall tower using a diamond trimming knife (trimtool 45, DiATOME, Hatfield, PA). Trimmed sample and entire stub were coated with gold palladium to assist in charge dissipation. The coated sample was then inserted into a serial block-face scanning electron microscope (VOLUMESCOPE Thermo Fisher Scientific, Inc., Waltham, MA) and allowed to acclimate to high vacuum for 12 hours prior to the start of imaging.

High resolution block-face images were obtained in a low vacuum environment using a beam energy of 3.0 kV with a current of 100 pA and a scanning dwell time of 2 μs and a 10-nm pixel size. A stack of approximately 500 block-face images were obtained while cutting the block at 50 nm increments. The image stack was then aligned and filtered using Amira software (Thermo Fisher Scientific, Inc., Waltham, MA) with further analysis performed using Reconstruct software (Fiala, J. C., 2005, J Microsc 218(1):52-61).

### Drug Release Assay

0.3 mL TISSEEL (Baxter healthcare Corp., Deerfield, IL) and TISSEEL-PEP were prepared and incubated in 2 mL PBS. The mixture was stored in a 37° C. incubator. The vesicle concentrations in the supernatant were measured using NANOSIGHT NS300 (Malvern Instruments, Malvern, UK) on day 1, day 3, day 7, day 14.

### RNA-Seq and Data Analysis

Wound tissue was harvested 28 days post operation. RNA was extracted using TRIZOL PLUS RNA purification kit (Thermo Fisher Scientific, Inc., Waltham, MA) according to the protocol. RNA library preparations and sequencing reactions were conducted at GENEWIZ, LLC. (South Plainfield, NJ). Data analysis was performed following the standard mRNA analysis pipeline. Expression levels of mRNAs were computed as normalized count number for Poisson-Distribution-based statistical analysis (Fang et al., 2012, Cell Biosci 2(1):26). Significantly changed genes (|log₂FC|>0.5 and p<0.05) with relatively high expression level (Normalized read counts>100) were used for heatmap visualization and further analysis. Gene Ontology (GO) and pathway enrichment analysis were performed by clusterProfiler (V3.12.0; Yu et al., 2012, OMICS 16(5):284-287) and heatmap visualization was conducted by pheatmap (V1.0.12; Luo, W and Brouwer, C., 2013, Bioinformatics 29(14):1830-1831).

### Statistical Analysis

Quantitative results were expressed as the mean value standard error. Statistical analysis was performed using two-way ANOVA and 2-tailed unpaired Student's t test (SPSS Statistics 13.0, SPSS Inc., Chicago, IL; Prism 8.0, GraphPad Software, San Diego, CA). A value of p<0.05 was considered statistically significant.

The complete disclosure of all patents, patent applications, publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of promoting healing of a wound, the method comprising administering to the wound an amount of a PEP preparation effective to promote healing of the wound.

2. The method of claim 1, wherein the PEP preparation comprises a hydrogel comprising a basement membrane protein.

3. The method of claim 1, wherein the PEP preparation comprises a hydrogel comprising a thrombin sealant or a fibrin sealant.

4. The method of claim 1, wherein the wound comprises an ischemic wound, a puncture wound, a laceration, an abrasion, a surgical wound, a skin graft, or a traumatic wound.

5. The method of claim 1, wherein the amount of PEP preparation is effective to increase angiogenesis, increase migration of fibroblasts into the wound, or increase migration of keratinocytes into the wound compared to a comparable untreated wound.

6. The method of claim 1, wherein the amount of PEP preparation is effective in donating TGF-β to increase expression COL1A or COL3A compared to a comparable untreated wound.

7. The method of claim 1, wherein the amount of PEP preparation is effective to decrease Wagner Ulcer Classification grade of the wound compared to a comparable untreated wound.

8. The method of claim 1, wherein the amount of PEP preparation is effective to decrease reaction force variation (R_{c}) or increase resistance to tensile force compared to a comparable untreated wound.

9. The method of claim 1, wherein the amount of PEP preparation is effective to increase expression of SMAD2, RAS, MKK3, RHOA, P38, or periostin in keratinocytes compared to untreated keratinocytes.

10. The method of claim 1, wherein the amount of PEP preparation is effective to increase expression of SMAD2, RAS, MKK3, ERK1, or TAK1 in fibroblasts compared to untreated fibroblasts.

11. The method of claim 2, wherein the wound comprises an ischemic wound, a puncture wound, a laceration, an abrasion, a surgical wound, a skin graft, or a traumatic wound.

12. The method of claim 2, wherein the amount of PEP preparation is effective to increase angiogenesis, increase migration of fibroblasts into the wound, or increase migration of keratinocytes into the wound compared to a comparable untreated wound.

13. The method of claim 2, wherein the amount of PEP preparation is effective to decrease Wagner Ulcer Classification grade of the wound compared to a comparable untreated wound.

14. The method of claim 3, wherein the wound comprises an ischemic wound, a puncture wound, a laceration, an abrasion, a surgical wound, a skin graft, or a traumatic wound.

15. The method of claim 3, wherein the amount of PEP preparation is effective to increase angiogenesis, increase migration of fibroblasts into the wound, or increase migration of keratinocytes into the wound compared to a comparable untreated wound.

16. The method of claim 3, wherein the amount of PEP preparation is effective to decrease Wagner Ulcer Classification grade of the wound compared to a comparable untreated wound.

17. The method of claim 4, wherein the amount of PEP preparation is effective to increase angiogenesis, increase migration of fibroblasts into the wound, or increase migration of keratinocytes into the wound compared to a comparable untreated wound.

18. The method of claim 4, wherein the amount of PEP preparation is effective to decrease Wagner Ulcer Classification grade of the wound compared to a comparable untreated wound.

19. The method of claim 11, wherein the amount of PEP preparation is effective to increase angiogenesis, increase migration of fibroblasts into the wound, or increase migration of keratinocytes into the wound compared to a comparable untreated wound.

20. The method of claim 11, wherein the amount of PEP preparation is effective to decrease Wagner Ulcer Classification grade of the wound compared to a comparable untreated wound.

21. The method of claim 14, wherein the amount of PEP preparation is effective to increase angiogenesis, increase migration of fibroblasts into the wound, or increase migration of keratinocytes into the wound compared to a comparable untreated wound.

22. The method of claim 14, wherein the amount of PEP preparation is effective to decrease Wagner Ulcer Classification grade of the wound compared to a comparable untreated wound.
